(19) United States
(12) Patent Application Publication
(10) Pub. No.: US 2014/0256032 A1
(43) Pub. Date: Sep. 11, 2014

(54) LIGHT SCATTERING SPERM ASSESMENT DEVICE AND METHOD

(71) Applicant: CHURCH & DWIGHT CO., INC., Princeton, NJ (US)

(72) Inventors: Nicholas James Wooder, Royston (GB); Giles Sanders, Fowlmere (GB); Roger Brian Minchin Clarke, Cambridge (GB); Albert R. Nazareth, Mercerville, NJ (US); Shang Li, West Windsor, NJ (US)

(73) Assignee: CHURCH & DWIGHT CO., INC., Princeton, NJ (US)

(21) Appl. No.: 14/183,909

(22) Filed: Feb. 19, 2014

Related U.S. Application Data
(60) Provisional application No. 61/774,960, filed on Mar. 8, 2013.

(51) Int. Cl. G01N 33/50

(52) U.S. Cl. CPC G01N 33/5005
USPC 435/288.7

(57) ABSTRACT

Test kits for assessing male fertility include a sample holder containing at least one sample chamber, a laser light source, and a light detector to detect scattered light intensity from the sample chamber. The sample holder may include multiple sample chambers connected by sperm swim channels. The test kit may have a housing with a maximum linear dimension of no more than 100 mm. Processing circuitry may be provided that is configured to produce a sperm count and/or sperm motility measurements by processing data from scattered light intensity measurements.

### REFERENCE TO RELATED APPLICATION

This application is related to U.S. Provisional Application Ser. No. 61/774,960 filed Mar. 8, 2013, and takes priority therefrom.

### BACKGROUND

1. Field

The embodiments disclosed herein relate to test devices and methods for assessing male fertility.

2. Description of the Related Art

Male fertility is generally assessed by counting the number of sperm per milliliter in a semen sample. Traditionally, this has been done manually by a trained andrologist. A semen sample is placed under a microscope, and the number of observed sperm are counted in a given area of view. This count is correlated to sample volume to produce a value for sperm per milliliter. In addition to sperm count, sperm motility is also a significant factor in assessing male fertility. A qualitative assessment of sperm motility can be made by visually evaluating the motion of the sperm in the sample under the microscope. These microscope systems are generally expensive, and can produce inconsistent results, even when used by well trained personnel.

Home use reagent based sperm count assays have been developed, such as the SpermCheck® male fertility test kit produced by ContraVac, Inc. and Princeton BioMeditech Corp. This kit can be used at home for a threshold test of sperm count. However, a numerical result for sperm count is not obtainable with this test kit, and it has no facility for assessing sperm motility.

### SUMMARY

Various implementations of devices and systems within the scope of the appended claims each have several aspects, no single one of which is solely responsible for the desirable attributes described herein. Without limiting the scope of the appended claims, some prominent features are described herein. Details of one or more implementations of the subject matter described in this specification are set forth in the accompanying drawings and the description below. Other features, aspects, and advantages will become apparent from the description, the drawings, and the claims. Note that the relative dimensions of the following figures may not be drawn to scale.

In one implementation, an apparatus for assessing male fertility, the apparatus comprises a first sample chamber having a perimeter. A plurality of sperm swim channels extend from different positions on the perimeter of the first sample chamber and terminate with a respective plurality of additional sample chambers separate from the first sample chamber and separate from each other. The apparatus also comprises one or more light sources, one or more light detectors positioned to detect scattered light from the first sample chamber and from at least two additional sample chambers when illuminated by one or more of the light sources, and a data processor, wherein the data processor is configured to produce a sperm count based at least in part on detected scattered light.

In another implementation, a sperm sample holder for measuring sperm motility comprises an entrance port configured to receive a semen sample, a first sample chamber having a perimeter and coupled to the entrance port, a plurality of sperm swim channels extending from different positions on the perimeter of the first sample chamber and terminating with a respective plurality of additional sample chambers separate from the first sample chamber and separate from each other. In some implementations, at least some of the sperm swim channels are of different lengths.

In another implementation, a system for measuring sperm motility comprises a sample holder comprising at least one sample chamber and configured to receive a semen sample, a housing having a maximum linear dimension of no more than 100 mm and wherein the ratios of height to width, height to length, and length to width are between 0.1 and 10, an opening in the housing configured to receive the sample holder, and a sample support contained within the housing adjacent to the opening. At least one light source contained within the housing is positioned to direct light along an axis that intersects at least one sample chamber when positioned in the sample support. At least one light detector contained within the housing is positioned to detect scattered light at a fixed scattering angle range from at least one sample chamber when positioned in the sample support. A data processor contained within the housing is coupled to the at least one light detector.

### BRIEF DESCRIPTION OF THE DRAWINGS

FIG. 1 is a block diagram of a male fertility assessment apparatus utilizing light scattering.

FIG. 2 is a graph comparing male fertility assessments made with an apparatus built according to the principles of FIG. 1 and manual andrologist assessment of male fertility on the same semen samples.

FIG. 3A is a block diagram of a male fertility assessment apparatus utilizing light scattering from multiple sample chambers.

FIG. 3B is a plan view of the sample holder with multiple sample chambers connected by swim channels of FIG. 3A.

FIG. 4 is a graph of idealized scattering amplitudes which may be obtained when using the male fertility assessment apparatus of FIG. 3A.

FIG. 5A is a plan view of another embodiment of a sample holder with multiple sample chambers connected by swim channels.

FIG. 5B is a plan view of another embodiment of a sample holder with multiple sample chambers connected by swim channels.

FIG. 6 is an illustration of a small size, low cost male fertility assessment apparatus.

FIG. 7 is a cross section of the apparatus of FIG. 6.

### DETAILED DESCRIPTION

The following detailed description is directed to certain implementations for the purposes of describing the innovative aspects. However, the teachings herein can be applied in a multitude of different ways.

Various aspects of implementations within the scope of the appended claims are described below. It should be apparent that the aspects described herein may be implemented in a wide variety of forms and that any specific structure and/or function described herein is merely illustrative. Based on the present disclosure a person/one having ordinary skill in the art should appreciate that an aspect described herein may be implemented independently of any other aspects and that two or more of these aspects may be combined in various ways. For example, an apparatus or system may be implemented or practiced using any number of the aspects set forth herein. In addition, such an apparatus or system may be implemented or practiced using other structure and/or functionality in addition to or other than one or more of the aspects set forth herein.

The word “illustrative” is used herein to mean “serving as an illustration, example, or instance.” Any implementation described herein as “illustrative” is not necessarily to be construed as preferred or advantageous over other implementations. The following description is presented to enable any person skilled in the art to make and use the invention. Details are set forth in the following description for purpose of explanation. It should be appreciated that one of ordinary skill in the art would realize that the invention may be practiced without the use of these specific details. In other instances, well known structures and processes are not elaborated in order not to obscure the description of the invention with unnecessary details. Thus, the present invention is not intended to be limited by the implementations shown, but is to be accorded with the widest scope consistent with the principles and features disclosed herein.

FIG. 1 is a block diagram of a system for assessing male fertility which utilizes light scattering by sperm 20 present in a sample chamber 12. A light source 14, preferably a coherent monochromatic light source 14, e.g. a laser, outputs a beam that may be collimated and that is incident on the sample chamber. Sperm 20 present in the sample chamber 12 scatter some of the incident light at an angle θ from the incident direction. The scattered light is incident on a light detector 16 such as a photodiode. The output of the detector is routed to a suitable system to quantify the output, e.g. an analog to digital converter 17, which provides a measure of the intensity of the scattered light at the scattering angle θ. The digitized intensity is routed to a data processor 18 which may process and store the data indicating scattered light intensity received by the detector 16. Within the scope of biological applicability, the more sperm there are in the sample chamber 12, the more light is scattered, and a larger output is produced by the light detector 16. The exact value of the scattering angle is not particularly significant to the relationship between sperm quantity and scattered light intensity, and may range from just a few degrees (e.g. less than 5 degrees) to 40 degrees or more. The incident beam need not be perpendicular to a face of the sample chamber. Although FIG. 1 shows a single scattering angle θ, it will be appreciated that physical light detectors 16 have an active area which will gather light over a range of angles Δθ, where Δθ will depend on the width of the detector active area and the relative positions and orientations of the sample chamber 12 and light detector 16. The system of FIG. 1 may therefore provide an indication of scattered light in a fixed scattering angle range of θ±Δθ. The value of Δθ may be small, less than 5 degrees, or less than 10% of θ, for example, and may be defined by optical elements positioned between the sample chamber 12 and light detector 16, rather than by the physical size of the active area of the light detector 16.

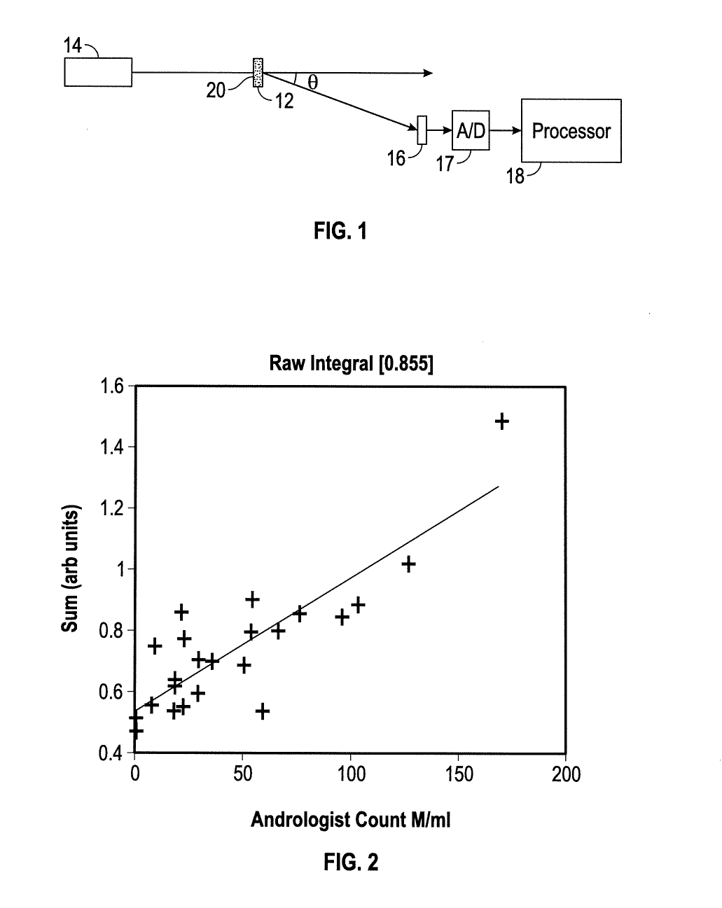

FIG. 2 shows a graph showing the correlation between scattered light intensity and andrologist sperm counts for a series of semen samples having varying sperm concentrations. The measured scattered light intensity is the vertical axis in arbitrary units, the andrologist count is the horizontal axis (million/ml, and each + represents a semen sample analyzed with both methods placed at the intersection of the apparatus measured scattered light intensity and andrologist manual count values. This graph shows a high correlation between scattered light intensity and andrologist produced manual counts, illustrating the high accuracy of the scattered light method for producing sperm count measurements.

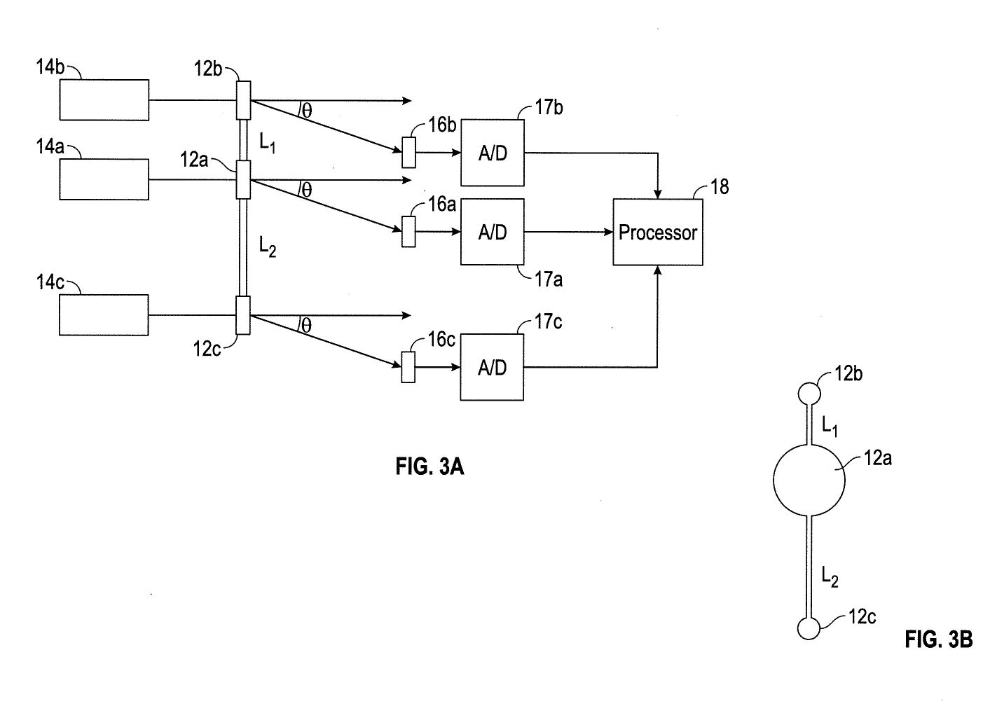

Although the system illustrated in FIG. 1 can measure the concentration of sperm in a sample, light will be scattered by both motile and non-motile sperm in the sample chamber, without the introduction of complex algorithms. Thus, the system of FIG. 1 does not distinguish live from dead sperm. For this reason, the sperm count measurement produced by the system of FIG. 1 is an incomplete measurement of fertility. A system and method for also obtaining motility information about sperm sample is illustrated in FIGS. 3A and 3B. In this system, multiple sample chambers are provided. These are illustrated in FIG. 3B. A first one of the chambers 12a, which may be referred to as a primary sample chamber, has a plurality of sperm swim channels extending from different positions and in different directions on its perimeter. Each of these swim channels terminates in an additional sample chamber 12b and 12c, which may be referred to as secondary sample chambers. These additional sample chambers are separate from the first sample chamber 12a and are separate from each other so as to contain potentially different sperm concentrations depending on the amount of migration of sperm within the swim channels. The chamber 12a may be initially loaded with the semen sample to be analyzed. Over time, motile sperm in the chamber 12a that was initially loaded with the semen sample will migrate down the swim channels to the other sample chambers 12b and 12c. Thus, the concentration of sperm in the chambers 12b and 12c will increase over time, providing a measure of the motility of the sperm that were initially loaded into the first chamber 12a.

The system for measuring the concentration of sperm in each of the sample chambers 12a, 12b, and 12c is illustrated in FIG. 3A. Essentially, this system includes a measurement system of FIG. 1 for each sample chamber. For the sample chamber design of FIG. 3B, the system includes three laser light sources 14a-14c, three light detectors 16a-16c, and three analog to digital converters 17a-17c. The outputs of the analog to digital converters are again routed to a processor circuit for analysis. The scattered light intensity from each sample chamber can be measured as a function of time and recorded by the processor. As noted above, faster increases in sperm count measured by the scattered light intensity from each of the additional chambers 12b and 12c correlate to higher sperm motilities.

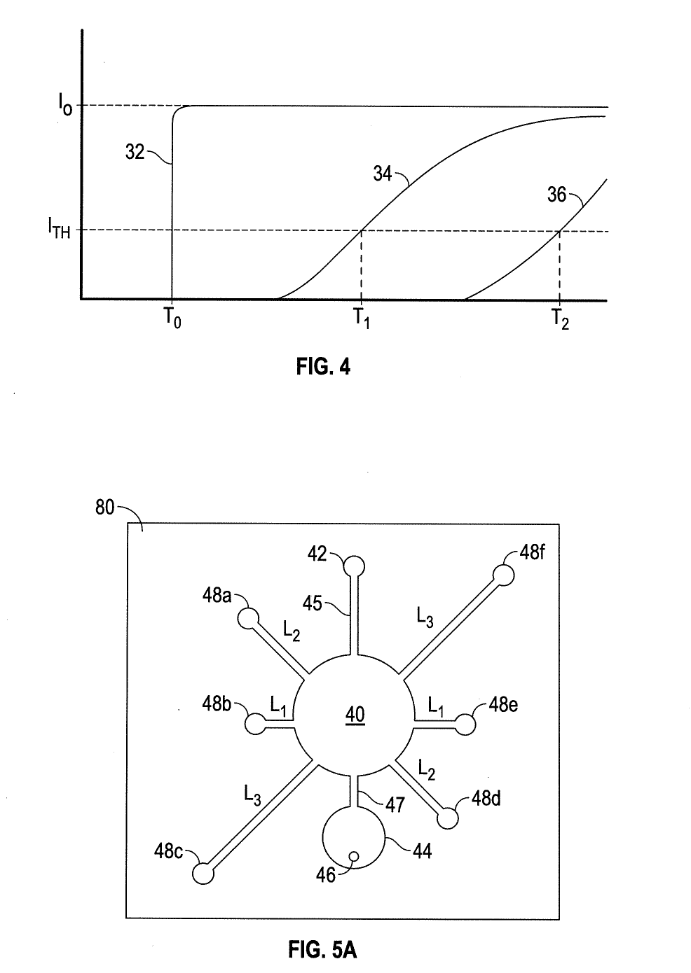

FIG. 4 illustrates example measurements that might be acquired for a sperm sample using the system of FIGS. 3A and 3B. As noted above, the scattered light intensity from each chamber will be dependent on the sperm concentration inside each sample chamber. In FIG. 4, curve 32 represents scattered light intensity form the primary sample chamber 12a. As the semen sample is initially loaded into this chamber, the scattered light intensity will essentially immediately go to an intensity I₀ at time T₀ when the sample is applied and initially placed under laser illumination. This scattered light intensity is dependent on the total sperm count in the sample. Initially, there will be no sperm in the secondary chambers, so the scattered light intensity from these chambers will be very low. After a time period related to the length of the swim channel connecting the primary chamber to a given secondary chamber, motile sperm will begin to appear in each secondary chamber. In the implementation shown in FIGS. 3A and 3B, sample chamber 12b is located a distance L₁ from the perimeter of the primary chamber 12a, and sample chamber 12c is located a distance L₂ from the perimeter of the primary chamber 12a, where L₁ is shorter than L₂. Curve 34 represents the scattered light intensity as a function of time from sample chamber 12b, and curve 36 represents the scattered light intensity as a function of time from sample chamber 12c. As shown in FIG. 4, the scattered light intensity from chamber 12b begins to increase before the scattered light intensity from chamber 12c begins to increase. The time from sample application to the time at which the sperm reach the secondary chambers and the rate at which the more sperm enter the secondary chambers is indicative of sperm motility. If the volume of the swim channels and secondary chambers is small compared to the primary chamber, then the scattered light intensity from the primary chamber will not change appreciably as motile sperm move toward the secondary chambers.

Sperm are typically graded by andrologists using a grading scale that classifies sperm into four categories. Grade a sperm swim fast in a straight line, Grade b sperm tend to swim forward but will swim in curved or crooked motions. Grade c sperm exhibit tail motion, but do not move appreciably. Grade d sperm show no activity at all. In a semen sample, the fraction of sperm as a percentage that are classified as Grade a or b is often referred to as the “progressive motility” of the sample and in addition to sperm count, is an important measure of fertility. The swim channels and multiple secondary sample chambers of the system of FIGS. 3A and 3B provides a good measure of this parameter as only Grade a and b sperm will be able to make their way down the swim channels to the secondary chambers.

To produce a numerical measure of progressive motility, the scattering data from the multiple chambers can be analyzed in a wide variety of ways. If the sizes of the sample chambers and swim channels are such that the motile sperm can freely swim in all directions to any chamber, it can be expected that after a final steady state of sperm concentration over the entire sample holder is reached, that the concentration of motile sperm in the secondary chambers will be the same as the concentration of motile sperm in the primary chamber. For example, if all of the sperm in the primary chamber are motile, after a long enough wait, the concentration of sperm in the secondary chambers will be the same as the concentration of sperm in the primary sample chamber. If the volume of the primary sample chamber is much larger than the volume of the swim channels and secondary chambers, the sperm concentration in the secondary chambers can be divided by the sperm concentration in the primary chamber (which will include both motile and non-motile sperm) for a measure of percentage Grade a and b sperm, which provides a measure of progressive motility. For this measurement, only a single secondary sample chamber is required, and the data analysis can essentially involve simply dividing the scattered light intensity from a secondary chamber by the scattered light intensity from the primary chamber.

Although the test described above may require a long wait time, sufficient information to produce a useful motility assessment may be available much faster. For example, as shown in FIG. 4, the time durations T₁-T₀ and T₂-T₀ at which the scattered light intensity reaches a threshold I_{TH} may be measured. The threshold I_{TH} may be set as a percentage of the primary chamber scattered light intensity I₀. The values L₁/(T₁−T₀) and L₂/(T₂−T₀) are measures of average or typical velocity of sperm swimming down each channel. These measurements and/or functions thereof may be correlated to progressive motility empirically using calibration testing with samples of known progressive motility values for use when testing a sample of unknown progressive motility. These methods may in some cases also work with a single secondary sample chamber, using multiple secondary sample chambers can increase the reliability of the result by providing checks for consistency of results and by providing more swim channel exits through which the motile sperm can exit the primary sample chamber.

Simpler data analysis can also be performed where the ultimate desired output is not a numerical measure of total sperm count and/or progressive motility, but is only a binary motile/not motile type of indication, for example.

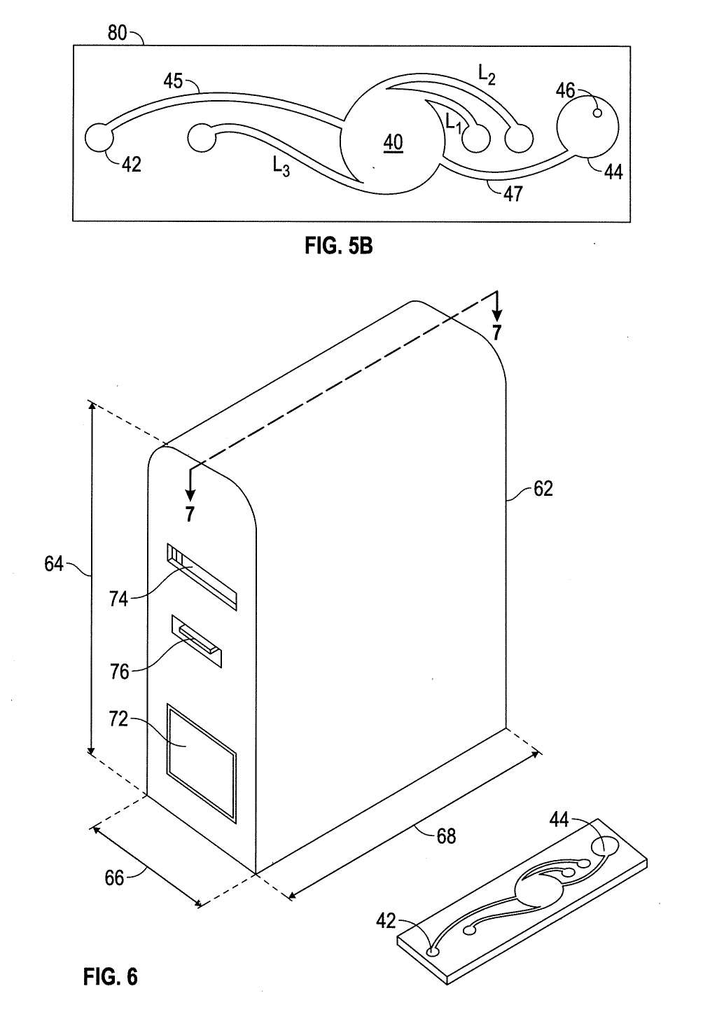

FIGS. 5A and 5B illustrate two designs for sample holders with a primary sample chamber and a plurality of secondary sample chambers. In these Figures, a sample holder 80, which may be a planar glass slide having length and width of about 10-80 mm with a 1-3 mm thickness, includes a primary chamber 40. The primary chamber 40 is connected to a sample input channel 45 that extends to a sample input port 42. Before the sample is loaded into the sample holder, the primary chamber 40, swim channels, and secondary chambers 48 may be filled with a saline or other solution. When the semen sample is injected into the sample holder via sample input port 42, the saline solution in the primary chamber 40 may exit the primary chamber 40 through an exit channel 47 that is coupled to a storage chamber 44 containing a vent 46. The storage chamber 44 may initially be free of fluid, and may have a sufficient volume to accept all of the saline solution originally in the primary sample chamber 40 as air is vented from the vent 46. This allows the semen sample to displace the saline solution from the primary sample chamber 40 while maintaining the saline solution in the swim channels and secondary sample chambers 48. FIG. 5A illustrates a substantially square sample holder with multiple linear swim channels extending in different directions radially from the primary chamber 40. FIG. 5B illustrates a rectangular sample holder with multiple curved swim channels extending in different more tangential directions from the perimeter of the primary sample chamber 40.

Since the swim velocity of motile sperm is typically between about 1 and 4 mm/minute, the length of the swim channels may advantageously be in the range of 1 to 40 mm to provide an opportunity for sperm to reach the secondary sample chambers in a time period that allows for a test time of no more than ten minutes, and in many cases less than five minutes.

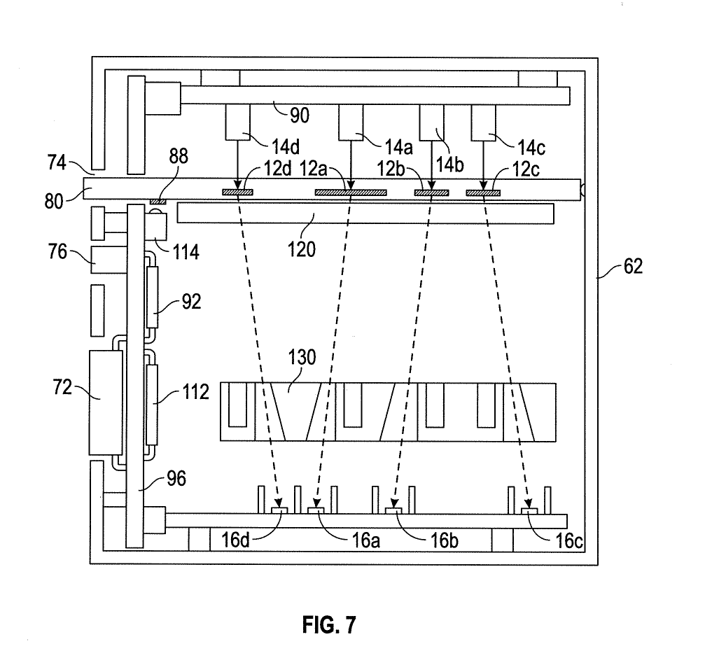

A male fertility test kit produced in accordance with the above principles is illustrated in FIGS. 6 and 7. FIG. 7 is a cross section of the apparatus of FIG. 6 along line 7 in FIG. 6. The apparatus includes a housing 62, which may be much smaller than any similar function apparatus previously produced. For example, the enclosure 62 may have a height 64 of 50 mm or less, a width 66 of 20 mm or less, and a length 68 of 50 mm or less. In this package, the maximum dimension of the housing from one corner to the farthest diagonal corner is less than 75 mm, and it has a volume of about 50,000 cubic millimeters. Preferably, the housing has a maximum dimension between two points of maximum linear distance apart of no more than 100 mm, and may be less than 50 mm depending on characteristics of the components used as described above. Also, the ratios of height 64 to width 66, height 64 to length 68, and length 68 to width 66 may be between 0.1 and 10. The volume is preferably less than 100,000 cubic millimeters, and may be less than 10,000 cubic millimeters.

The apparatus includes a sample input port 74, which is configured to accept a sample slide 80 having the sample chambers and swim channels embedded therein. The sample slide 80 may also have a reflective portion 88 for automated start-up of the apparatus when the sample slide 80 is inserted as described further below.

The apparatus may include a display 72, such as an LCD display for outputting results of the fertility assessment. One or more LED lights may additionally or alternatively be provided for outputting assessment results. In addition, a digital output or input/output port 76 may be provided for outputting results to a separate computing device such as a PC. This may be a USB port for example. This port may be used to communicate results, raw image data, or other information generated by the apparatus during use. The port 76 may also be used to input image processing parameters or other functional instructions to the apparatus. The same communication capability could also be provided wirelessly.

Turning now to FIG. 7, the internal components of the apparatus are illustrated with the sample slide 80 inserted. As described above with reference to FIGS. 1, 3A, and 3B, the sample chambers 12a-12d are positioned adjacent to laser light sources 14a-14d. On the other side of the sample chambers 12a-12d, light sensors 16a-16d receive scattered light from each respective sample chamber. In this implementation, there are the same number of laser sources, sample chambers, and light detectors. Although a variety of packaging options could be used, this implementation includes a printed circuit board 90 mounted to the top of the housing 62 which mounts the laser light sources. A second printed circuit board 96 connected to the top printed circuit board 90 is coupled to the front of the housing 62 and mounts the display 72, processor integrated circuit 112, and data port 76. An opening may be provided in this printed circuit board 96 through which the sample slide 80 is inserted. This circuit board 96 may also mount an LED and photodetector 114 that is adjacent to the reflective portion 88 of the sample slide 80 when the slide 80 is fully seated in the sample input port 74. When the photodetector receives reflected LED light, the system may automatically exit a sleep mode and begin performing light scattering measurements.

In the implementation of FIGS. 6 and 7, the light sources 14a-14d are provided in a planar array, and the sample holder includes sample chambers 12a-12d in a planar array. In the implementation of FIGS. 6 and 7, the relative positions of the light sources are substantially the same as the relative positions of the sample chambers. The light detectors 16a-16d are also provided in a planar array that is parallel to the planar array of light sources 14a-14d. The device can be very compact, with the perpendicular distance between the planar array of light sources and the planar array of light detectors (measured from the exit apertures of the laser diodes to the active surfaces of the light detectors) being less than 50 mm in many advantageous embodiments.

It is one advantageous aspect of this device that it can be made inexpensively and of a small size. The laser light sources 14a-14d can be commercially available inexpensive laser diode light sources. An anamorphic beam collimation lens can be provided at the laser diode output. Some commercially available devices include integral collimation lenses and can produce a small circular spot size of about 1 or 2 mm with a very small beam divergence of much less than 1 degree. In some cases, collimating optics may not be required if the light detectors 16a-16d are oriented with respect to the laser diodes such that the scattering angle of the detected light is parallel to the width of the diode active region where the natural beam divergence of the laser diode is relatively small. Laser output powers of 10 mW or less, or even 5 mW or less may be used. The laser light sources may have a diameter less than 10 mm, and a length less than 20 mm, including collimating optics.

The light detectors 16a-16d can be small, inexpensive, commercially available photodiodes. They may measure less than 10 mm in length, width, and height.

As noted above, the scattering angle can vary, and may be selected to provide sufficient separation of the light detectors. A light shield 130 may be provided between the sample chambers 12a-12d and the light detectors 16a-16d to reduce stray scattered light from interfering with the measurements. The light shield 130 may be a polymer, may be black or otherwise light absorbing, and may include light passageways along the desired scattering angle between each sample chamber and its respective light detector. The light shield may also include blind holes as beam dumps for the unscattered beam. The light shield may also include lenses or other optics to focus the desired scattered light onto the active surface of the appropriate detector.

Although the implementation of FIGS. 6 and 7 includes multiple light sources and multiple light detectors, a single light source may be used with beam splitting and optical components to guide light to the appropriate sample chamber. Also, the multiple light detectors could be implemented as a single array of CCD elements, where scattered light from different sample chambers is incident on different portions of the array for separate measurements. As another alternative, a single light source and detector could be used while the sample holder is moved, or optical components are adjusted to take measurements from different chambers serially using a common light source and detector.

The foregoing description details certain embodiments of the invention. It will be appreciated, however, that no matter how detailed the foregoing appears in text, the invention can be practiced in many ways. As is also stated above, it should be noted that the use of particular terminology when describing certain features or aspects of the invention should not be taken to imply that the terminology is being re-defined herein to be restricted to including any specific characteristics of the features or aspects of the invention with which that terminology is associated. The scope of the invention should therefore be construed in accordance with the appended claims and any equivalents thereof.

What is claimed is:

1. An apparatus for assessing male fertility, the apparatus comprising:
a first sample chamber having a perimeter;
a plurality of sperm swim channels extending from different positions on the perimeter of the first sample chamber and terminating with a respective plurality of additional sample chambers separate from the first sample chamber and separate from each other;
one or more light sources;
one or more light detectors positioned to detect scattered light from the first sample chamber and from at least two additional sample chambers when illuminated by one or more of the light sources; and
a data processor, wherein the data processor is configured to produce a sperm count based at least in part on detected scattered light.

2. The apparatus of claim 1, wherein the light detectors are positioned to detect scattered light along a single off-axis scattering angle range.

3. The apparatus of claim 1, comprising a plurality of light sources and a corresponding plurality of light detectors.

4. The apparatus of claim 3, comprising the same number of light sources, light detectors, and sample chambers.

5. The apparatus of claim 1, wherein at least some of the sperm swim channels are different lengths.

6. The apparatus of claim 1, wherein at least some of the sperm swim channels extend in different directions from the first sample chamber.

7. A semen sample holder for measuring sperm motility, the holder comprising:
an entrance port configured to receive a semen sample; and
a first sample chamber having a perimeter and coupled to the entrance port;
a plurality of sperm swim channels extending from different positions on the perimeter of the first sample chamber and terminating with a respective plurality of additional sample chambers separate from the first sample chamber and separate from each other.

8. The semen sample holder of claim 7, wherein at least some of the sperm swim channels are different lengths.

9. The semen sample holder of claim 8, wherein each swim channel has a length between 1 and 40 mm.

10. The semen sample holder of claim 7, wherein at least some of the sperm swim channels extend in different directions from the first sample chamber.

11. A system for measuring sperm motility, the system comprising:
a sample holder comprising at least one sample chamber and configured to receive a semen sample;
a housing having a maximum linear dimension of no more than 100 mm and wherein the ratios of height to width, height to length, and length to width are between 0.1 and 10;
an opening in the housing configured to receive the sample holder;
a sample support contained within the housing adjacent to the opening;
at least one light source contained within the housing and positioned to direct light along an axis that intersects at least one sample chamber when positioned in the sample support;
at least one light detector contained within the housing and positioned to detect scattered light at a fixed scattering angle range from at least one sample chamber when positioned in the sample support; and
a data processor contained within the housing and coupled to the at least one light detector.

12. The system of claim 11, wherein the sample holder comprises a plurality of separate sample chambers.

13. The system of claim 12, comprising a plurality of light sources and a corresponding plurality of light detectors.

14. The system of claim 13, comprising the same number of light sources, light detectors, and sample chambers.

15. The system of claim 14, comprising:
a planar array of sample chambers on the sample holder defining a set of relative sample chamber positions;
a planar array of light sources defining a set of relative light source positions that are substantially the same as the set of relative sample chamber positions; and
a planar array of light detectors.

16. The system of claim 15, wherein the planar array of light sources is parallel to the planar array of light detectors.

17. The system of claim 16, wherein the perpendicular distance between the planar array of light sources and the planar array of light detectors is 50 mm or less.

18. The system of claim 15, wherein the planar array of light detectors defines a set of relative light detector positions that are substantially the same as the set of relative sample chamber positions.

19. The system of claim 16, comprising a planar array of optical components positioned between and parallel to the planar array of light sources and the planar array of light detectors.

20. The system of claim 12, wherein the sample holder comprises:
an entrance port configured to receive a semen sample; and
a first sample chamber having a perimeter and coupled to the entrance port;
a plurality of sperm swim channels extending from different positions on the perimeter of the first sample chamber and terminating with a respective plurality of additional sample chambers separate from the first sample chamber and separate from each other.

21. The system of claim 20, comprising:
a planar array of light sources positioned on one side of the sample support, the number of light sources being the same as the number of sample chambers;
a planar array of light detectors parallel to the planar array of light sources and positioned on an opposite side of the sample support; the number of light detectors being the same as the number of sample chambers; and
wherein the perpendicular distance between the planar array of light sources and the planar array of light detectors is 50 mm or less.
